(19) United States
(12) Patent Application Publication
(10) Pub. No.: US 2013/0184524 A1
(43) Pub. Date: Jul. 18, 2013

(54) Scanning Endoscope Device

(71) Applicant: OLYMPUS MEDICAL SYSTEMS CORP., Tokyo (JP)

(72) Inventors: Tomoko Shimada, Tokyo (JP); Masahiro Yoshino, Tokyo (JP); Makoto Igarashi, Tokyo (JP)

(73) Assignee: OLYMPUS MEDICAL SYSTEMS CORP., Tokyo (JP)

(21) Appl. No.: 13/673,290

(22) Filed: Nov. 9, 2012

Related U.S. Application Data
(63) Continuation of application No. PCT/JP2012/055186, filed on Mar. 1, 2012.

(30) Foreign Application Priority Data
Mar. 31, 2011 (JP) 2011-080634

(51) Int. Cl. A61B 1/06

(52) U.S. Cl. CPC A61B 1/0638
USPC 600/109

(57) ABSTRACT

A scanning endoscope device including two core portions that are provided parallel to each other and that radiate illuminating beams having optical characteristics different from each other toward a subject; a driving unit that two-dimensionally scans the two illuminating beams radiated from the core portions by causing vibration of distal-end portions of the core portions; a light receiving unit that receives return beams, returned from the subject, of the two illuminating beams; a light splitting unit that splits the return beams received by the light receiving unit according to the optical characteristics; two light detecting units that photoelectrically convert the two return beams split by the light splitting unit to output captured image signals; and an image generating unit that generates two images for two viewpoint based on the each captured image signal.

### CROSS-REFERENCE TO RELATED APPLICATIONS

This is a continuation of International Application PCT/JP2012/055186, with an international filing date of Mar. 1, 2012, which is hereby incorporated by reference herein in its entirety. This application claims the benefit of Japanese Patent Application No. 2011-080634, the content of which is incorporated herein by reference.

### TECHNICAL FIELD

The present invention relates to scanning endoscope devices.

### BACKGROUND ART

Conventional scanning endoscope devices that obtain two images from different viewpoints (parallax images) by irradiating mutually displaced points on an observation target with two light beams while two-dimensionally scanning the light beams are known (e.g., see Patent Document 1). It is possible to stereoscopically view the observation target by using such parallax images. In the case of Patent Document 1, actuators for scanning light beams are provided, one for each light beam, at the distal-end portion of an inserted portion.

### CITATION LIST

### Patent Literature

### {PTL 1}

Specification of U.S. Patent Application Publication No. 2009/0137893

### SUMMARY OF INVENTION

### Solution to Problem

The present invention provides a scanning endoscope device that obtains a parallax image, including a first core portion that radiates an illuminating light beam for a first viewpoint toward a subject, the illuminating light beam having a first optical characteristic; a second core portion that is provided parallel to the first core portion and that radiates an illuminating light beam for a second viewpoint toward the subject, the illuminating light beam having a second optical characteristic different from the first optical characteristic; a driving unit that two-dimensionally scans the illuminating light beam radiated from the first core portion and the illuminating light beam radiated from the second core portion by causing vibration of distal-end portions of the first core portion and the second core portion; a light receiving unit that receives return light beams, returned from the subject, of the illuminating light beam radiated from the first core portion and the illuminating light beam radiated from the second core portion; a light splitting unit that splits the return light beams received by the light receiving unit into a return light beam having the first optical characteristic and a return light beam having the second optical characteristic; a first light detecting unit that photoelectrically converts the return light beam split by the light splitting unit and having the first optical characteristic to output a first captured image signal for the first viewpoint; a second light detecting unit that photoelectrically converts the return light beam split by the light splitting unit and having the second optical characteristic to output a second captured image signal for the second viewpoint; and an image generating unit that generates a first image for the first viewpoint based on the first captured image signal output from the first light detecting unit and that generates a second image for the second viewpoint based on the second captured image signal output from the second light detecting unit.

### BRIEF DESCRIPTION OF DRAWINGS

{FIG. 1}

FIG. 1 is an overall construction diagram of a scanning endoscope device according to an embodiment of the present invention.

{FIG. 2}

FIG. 2 is an enlarged view of the distal-end portions of light-emitting fibers in FIG. 1.

{FIG. 3}

FIG. 3 is an illustration showing the distal-end face of an inserted portion in FIG. 1.

{FIG. 4}

FIG. 4 is a diagram showing two scanning areas where illuminating light beams are scanned by the scanning endoscope device in FIG. 1.

{FIG. 5}

FIG. 5 is an illustration showing a modification of the light-emitting fibers in FIG. 1.

{FIG. 6}

FIG. 6 is an illustration showing a construction in which GRIN lenses are provided at the distal-end faces of the light-emitting fibers in FIG. 2.

{FIG. 7}

FIG. 7 is an illustration showing a construction in which ball lenses are provided at the distal-end faces of the light-emitting fibers in FIG. 2.

### DESCRIPTION OF EMBODIMENTS

A scanning endoscope device 1 according to an embodiment of the present invention will be described below with reference to the drawings.

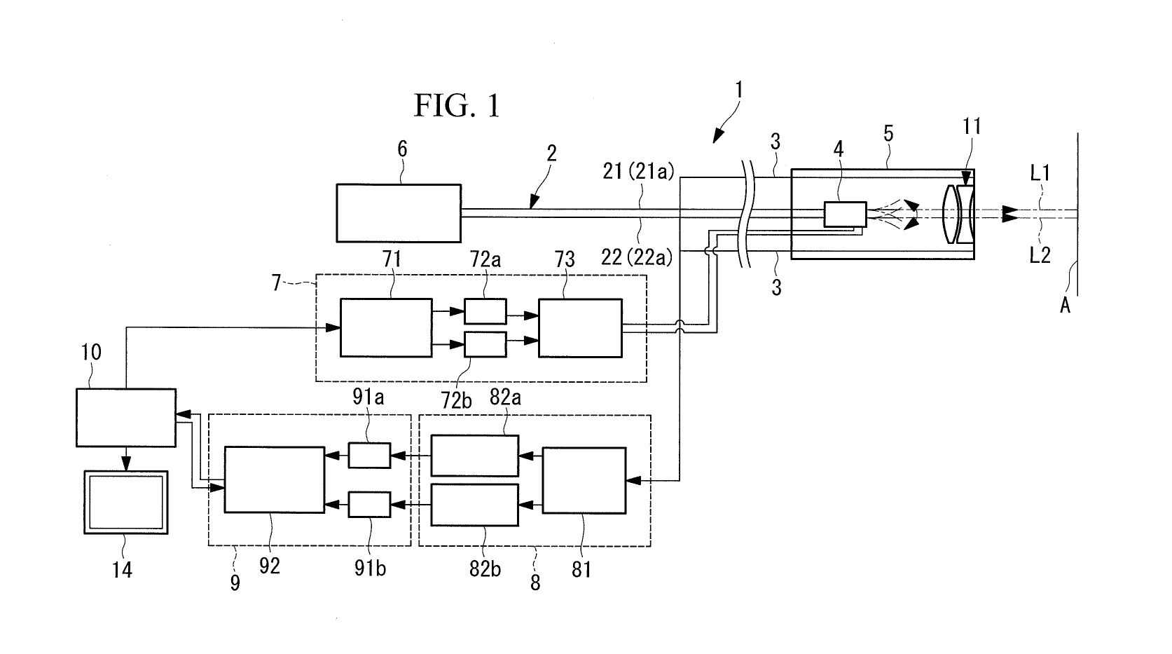

The scanning endoscope device 1 according to this embodiment obtains parallax images that enable stereoscopic viewing by the parallel method. As shown in FIG. 1, the scanning endoscope device 1 includes an inserted portion 5 having light-emitting fibers (optical fiber component) 2 that emit illuminating light beams L1 and L2, light-receiving fibers 3, and an actuator (driving unit) 4 that causes vibration of the distal-end portions of the light-emitting fibers 2; an illumination unit 6 that supplies the illuminating light beams L1 and L2 to the light-emitting fibers 2; a driving unit 7 that drives the actuator 4; a detection unit (detecting unit) 8 that performs photoelectric conversion of return light beams of the illuminating light beams L1 and L2 received by the light-receiving fibers 3; an image generation unit 9 that generates parallax images based on signals from the detection unit 8; and a control unit 10 that controls the operation of the illumination unit 6 and the driving unit 7 and that outputs the parallax images generated by the image generation unit 9 to a monitor 14.

The light-emitting fibers 2 and the light-receiving fibers 3 are disposed along the lengthwise direction inside the inserted portion 5. At the distal end of the light-emitting fibers 2, an illumination optical system 11 is provided.

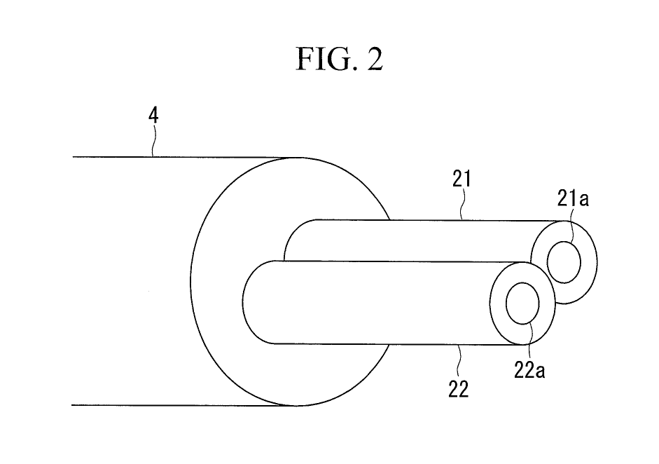

As shown in FIG. 2, the light-emitting fibers 2 include two optical fibers 21 and 22 that are joined together at least at their distal-end portions. The optical fibers 21 and 22 are single-mode fibers having cores (core portions) 21a and 22a, respectively. A first illuminating light beam L1 emitted from one core 21a and a second illuminating light beam L2 emitted from the other core 22a are condensed by the illumination optical system 11 and irradiate an observation surface A.

Here, as will be described later, the wavelength of the first illuminating light beam L1 and the wavelength of the second illuminating light beam L2 mutually differ. Therefore, because of aberrations that arise when these illuminating light beams L1 and L2 pass through the illumination optical system 11, the illuminating light beams L1 and L2 irradiate points on the observation surface A that are displaced in a direction crossing the optical axes.

At this time, preferably, the displacement d between the two illuminating light beams L1 and L2 is, for example, greater than or equal to about 80 μm and less than or equal to about 500 μm. Considering the diameter of each of the optical fibers 21 and 22, it is difficult to make the displacement d between the irradiated points less than 80 μm. On the other hand, a displacement d between the irradiated points greater than 500 μm is undesirable since the diameter of the inserted portion 5 becomes large. The displacement d between the irradiated points can also be designed by adjusting the distance between the two cores 21a and 22a, the emitting directions of the illuminating light beams L1 and L2 from the individual cores 21a and 22a, etc.

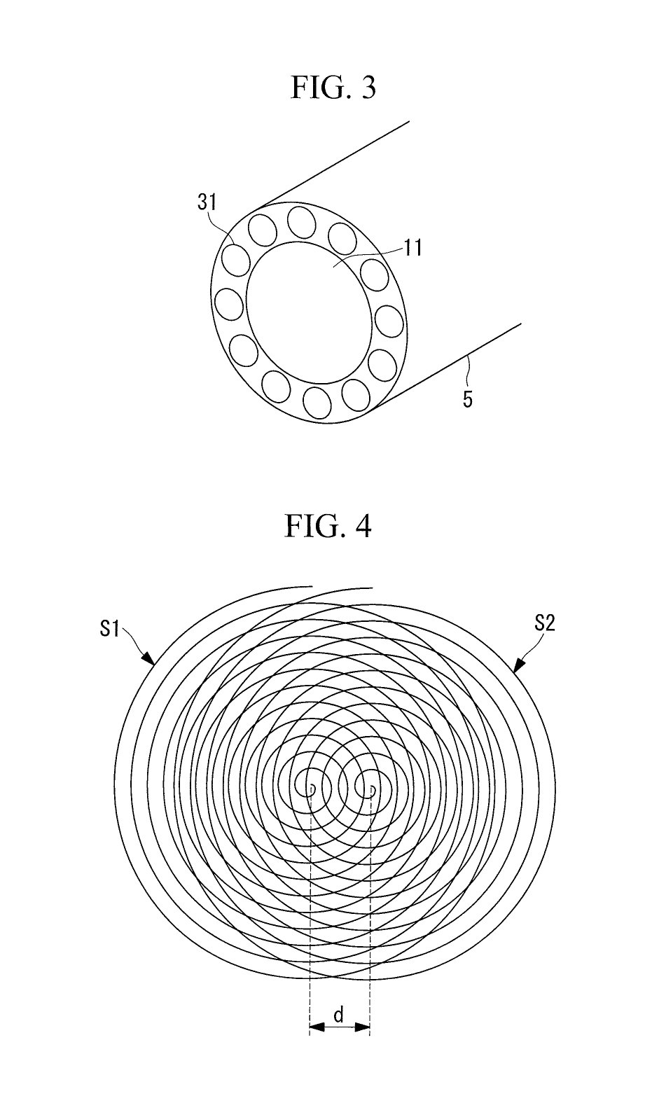

The light-receiving fibers 3 commonly receive return light beams of the two illuminating light beams L1 and L2 with light receiving faces (light receiving unit) 31 formed of the distal-end faces thereof and guide the received return light beams to the detection unit 8. Here, as shown in FIG. 3, multiple (12 in the example shown in the figure) light-receiving fibers 3 are provided, and the light receiving faces 31 are arranged to surround the illumination optical system 11 in the circumferential direction on the distal-end face of the inserted portion 5. This serves to increase the intensity of the return light received from the observation surface A.

The actuator 4 is, for example, an electromagnetic or piezoelectric actuator. When driving voltages (described later) are applied from the driving unit 7, the actuator 4 causes the distal-end portions of the light-emitting fibers 2 to vibrate in the directions of two axes (X direction and Y direction) crossing the lengthwise direction of the light-emitting fibers 2. Thus, the two illuminating light beams L1 and L2 are simultaneously scanned two-dimensionally on the observation surface A. There is no particular limitation about the scanning method, and spiral scanning, raster scanning, etc. can be used.

Here, since the distal-end portions of the two optical fibers 21 and 22 are joined together, the scanning trajectories of the two illuminating light beams L1 and L2 have the same shape, as shown in FIG. 4. Furthermore, scanning areas S1 and S2 (areas scanned by spiral scanning in the example shown in the figure) on the observation surface A scanned with the two illuminating light beams L1 and L2 are displaced by the displacement d between the points irradiated with the two illuminating light beams L1 and L2.

The illumination unit 6 is constructed to make the first illuminating light beam L1 having a first wavelength incident on one core 21a and to make the second illuminating light beam L2 having a second wavelength, which differs from the first wavelength, incident on the other core 22a. The first illuminating light beam L1 and the second illuminating light beam L2 are single-wavelength continuous-wave light. The first wavelength and the second wavelength are, for example, 532 nm and 440 nm. The illumination unit 6 is constructed of, for example, two light sources that individually emit the first illuminating light beam L1 and the second illuminating light beam L2. As the light sources, single-wavelength solid-state lasers, which have superior light guiding efficiency, are preferable.

The driving unit 7 includes a signal generator 71 that generates driving signals for driving the actuator 4 in the form of digital signals, D/A converters 72a and 72b that convert the driving signals generated by the signal generator 71 into analog signals, and a signal amplifier 73 that amplifies outputs of the D/A converters 72a and 72b.

The signal generator 71 generates two driving signals for vibrating the light-emitting fibers 2 in the X direction and Y direction and inputs the two driving signals to the separate D/A converters 72a and 72b. The signal amplifier 73 amplifies the analog signals generated by the D/A converters 72a and 72b, i.e., driving voltages, to an amplitude suitable for driving the actuator 4 and outputs the amplified driving voltages to the actuator 4.

The detecting unit 8 includes a wavelength splitter (wavelength splitting mechanism) 81 that splits return light beams guided by the individual light-receiving fibers 3 on the basis of their wavelengths and two light detectors 82a and 82b that detect the individual return light beams split by the wavelength splitter 81 and that performs photoelectric conversion.

The wavelength splitter (wavelength splitting unit) 81 extracts a return light beam having the first wavelength and a return light beam having the second wavelength among the input return light beams and outputs these return light beams to the separate light detectors 82a and 82b.

The light detectors (light detecting unit) 82a and 82b are, for example, photodiodes or photomultiplier tubes. The light detectors 82a and 82b output photocurrents having magnitudes corresponding to the intensities of the detected return light beams to A/D converters 91a and 91b, respectively.

The image generation unit 9 includes two A/D converters 91a and 91b that convert the photocurrents output from the individual light detectors 82a and 82b into digital signals and a parallax-image generator 92 that generates two-dimensional images from the digital signals generated by the individual A/D converters 91a and 91b.

The parallax-image generator 92 generates two two-dimensional images based on the digital signals received from the individual A/D converters 91a and 91b and information about the scanning positions of the illuminating light beams L1 and L2 (described later) received from the control unit 10. Here, the two two-dimensional images are an image generated from the return light beam from the scanning area S1 scanned with the first illuminating light beam L1 and an image generated from the return light beam from the scanning area S2 scanned with the second illuminating light beam L2. That is, the two two-dimensional images are images whose viewpoints are shifted in parallel by an amount corresponding to the displacement d between the points irradiated with the two illuminating light beams L1 and L2. It is possible to construct a parallax image from these two two-dimensional images.

The control unit 10 outputs specification signals giving the specifications of the driving signals, e.g., the frequency, amplitude, etc., to the signal generator 71 and outputs information about the specification signals, i.e., information including the scanning positions of the illuminating light beams L1 and L2, to the parallax-image generator 92.

Furthermore, the control unit 10 reconstructs an image suitable for stereoscopic observation from the two two-dimensional images received from the parallax-image generator 92 and displays the reconstructed image on the monitor 14. This enables an operator to stereoscopically observe an image of the observation surface A generated by the scanning endoscope device 1.

In this case, according to this embodiment, even though the construction is such that parallax images are obtained by using the two illuminating light beams L1 and L2, the single actuator 4 suffices to scan the two illuminating light beams L1 and L2, so that an advantage is afforded in that the diameter of the inserted portion 5 can be made small. Furthermore, since images of the observation surface A are obtained by using the illuminating light beams L1 and L2 having different wavelengths, it becomes possible to perform simultaneous observation using light beams in different wavelength ranges. For example, by modifying the first illuminating light beam L1 to an excitation light beam for a fluorescent pigment (e.g., a near-infrared light beam), modifying the second excitation light beam L2 to a white light beam in which light beams from three solid-state lasers for RGB are combined, and suitably modifying the wavelengths of the return light beams split by the wavelength splitter 81, it becomes possible to simultaneously observe a fluorescence image and a white-light image.

Although the illuminating light beams L1 and L2 radiated from the individual cores 21a and 22a have mutually different wavelengths in this embodiment, alternatively, the illuminating light beams L1 and L2 may have mutually different polarization directions. In this case, the illumination unit 6 includes, for example, two polarizers that extract light beams having different polarization directions and that output the light beams to the individual cores 21a and 22a. Furthermore, a polarized-light splitter (not shown, polarized-light splitting mechanism) that extracts light beams having the individual polarization directions is provided between the observation surface A and the light receiving faces 31.

Also with this construction, it is possible to separately detect return light beams from the individual scanning areas S1 and S2 and to separately generate images of the individual scanning areas S1 and S2. Furthermore, it becomes possible to use light beams having the same wavelength as the first illuminating light beam L1 and the second illuminating light beam L2.

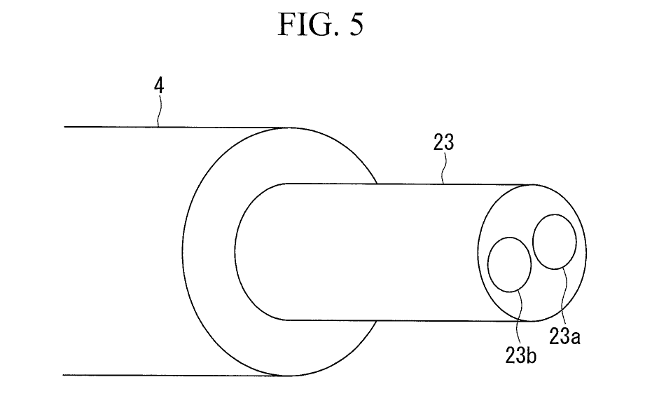

Furthermore, although the light-emitting fibers 2 include the two optical fibers 21 and 22 having a single core in this embodiment, alternatively, the light-emitting fiber 2 may consist of a single optical fiber 23 having two cores 23a and 23b, as shown in FIG. 5.

Also with this construction, it is possible to obtain parallax images by two-dimensionally scanning two illuminating light beams irradiating points that are displaced in a direction crossing the optical axes, simultaneously by means of the single actuator 4.

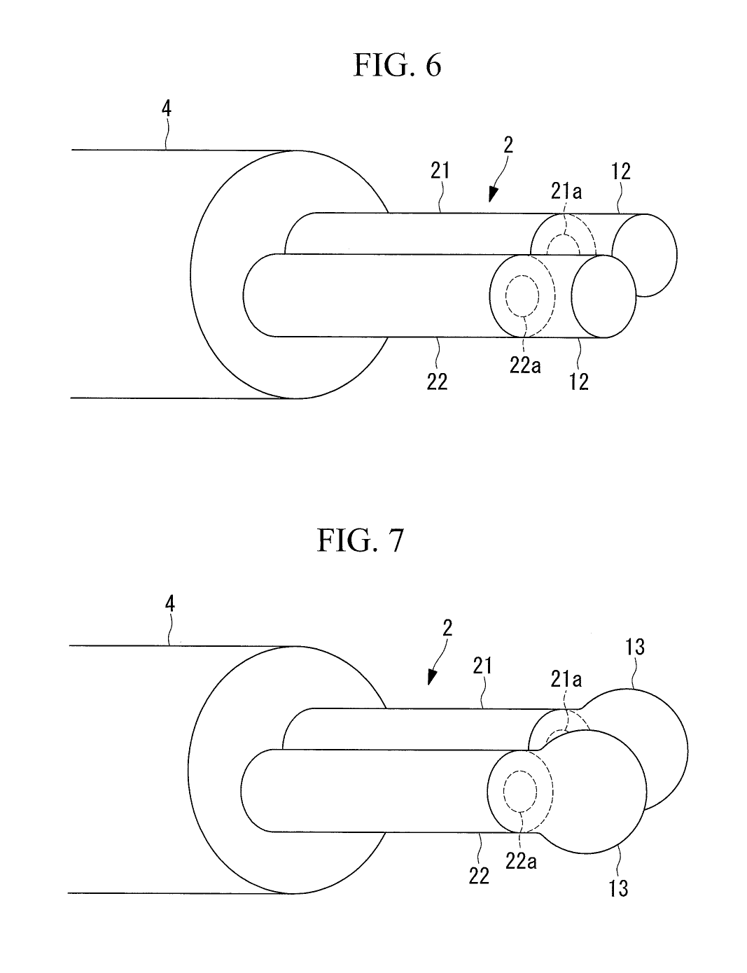

Furthermore, in this embodiment, optical components that condense the illuminating light beams L1 and L2 emitted from the individual cores 21a and 22a into collimated light beams or into smaller spot diameters may be joined at the distal-end faces of the two optical fibers 21 and 22. As the optical components, for example, GRIN (gradient index) lenses 12, shown in FIG. 6, or ball lenses 13, shown in FIG. 7, are used. This serves to improve the resolution of the parallax images. In the case where optical components are provided as described above, the illumination optical system 11 may be omitted.

Furthermore, although continuous light beams are used as the illuminating light beams L1 and L2 in this embodiment, alternatively, pulsed light beams may be used.

With this construction, since the cumulative irradiation periods of the observation surface A with the illuminating light beams L1 and L2 become shorter, the effects exerted on the observation surface A by the illuminating light beams L1 and L2 can be alleviated. For example, in the case of fluorescence observation, fading of the fluorescent pigment can be prevented. Furthermore, in the case where the observation surface A is irradiated with the first illuminating light beam L1 and the second illuminating light beam L2 in a time-division multiplexing, it is possible to perform time-resolved measurement of the behavior of biological molecules, etc. on the observation surface A.

In the case where pulsed light beams are used as the illuminating light beams L1 and L2, the illumination unit 6 may be constructed to make the two illuminating light beams L1 and L2 incident on the individual cores 21a and 22a at pulse timings shifted from each other, and the detection unit 8 may be constructed to detect return light beams in synchronization with the pulse timings. In this construction, the wavelengths of the illuminating light beams L1 and L2 may be either the same or different. The latter case is suitable for fluorescence imaging using two different fluorescent pigments.

Furthermore, although the light-emitting fibers 2 include the two cores 21a and 22a in this embodiment, alternatively, the light-emitting fibers 2 may include three or more cores. For example, even in the case where the distal-end portions of three or more optical fibers having a single core are joined together, the single actuator 4 suffices to scan illuminating light beams from all the cores. Therefore, it is possible to obtain images of the observation surface A by using three or more illuminating light beams while making the diameter of the inserted portion 5 small.

### REFERENCE SIGNS LIST

1 Scanning endoscope device
2 Light-emitting fibers
3 Light-receiving fibers
4 Actuator (driving unit)
5 Inserted portion
6 Illumination unit (illuminating unit)
7 Driving unit
8 Detection unit (detecting unit)
9 Image generation unit (image generating unit)
10 Control unit
11 Illumination optical system
12 GRIN lenses (optical components)
13 Ball lenses (optical components)
14 Monitor
21, 22, 23 Optical fibers (optical fiber component)
21a, 22a, 23a, 23b Cores (core portions)
31 Light receiving faces (light receiving unit)
71 Signal generator
72a, 72b D/A converters
73 Signal amplifier
81 Wavelength splitter (wavelength splitting mechanism)
82a, 82b Light detectors
91a, 91b A/D converters
92 Parallax-image generator
A Observation surface
L1 First illuminating light beam
L2 Second illuminating light beam

1. A scanning endoscope device that obtains a parallax image, comprising:
a first core portion that radiates an illuminating light beam for a first viewpoint toward a subject, the illuminating light beam having a first optical characteristic;
a second core portion that is provided parallel to the first core portion and that radiates an illuminating light beam for a second viewpoint toward the subject, the illuminating light beam having a second optical characteristic different from the first optical characteristic;
a driving unit that two-dimensionally scans the illuminating light beam radiated from the first core portion and the illuminating light beam radiated from the second core portion by causing vibration of distal-end portions of the first core portion and the second core portion;
a light receiving unit that receives return light beams, returned from the subject, of the illuminating light beam radiated from the first core portion and the illuminating light beam radiated from the second core portion;
a light splitting unit that splits the return light beams received by the light receiving unit into a return light beam having the first optical characteristic and a return light beam having the second optical characteristic;
a first light detecting unit that photoelectrically converts the return light beam split by the light splitting unit and having the first optical characteristic to output a first captured image signal for the first viewpoint;
a second light detecting unit that photoelectrically converts the return light beam split by the light splitting unit and having the second optical characteristic to output a second captured image signal for the second viewpoint; and
an image generating unit that generates a first image for the first viewpoint based on the first captured image signal output from the first light detecting unit and that generates a second image for the second viewpoint based on the second captured image signal output from the second light detecting unit.

2. A scanning endoscope device according to claim 1, wherein the first optical characteristic is a first wavelength range, and the second optical characteristic is a second wavelength range, which differs from the first wavelength range.

3. A scanning endoscope device according to claim 1, wherein the first optical characteristic is a first polarization direction, and the second optical characteristic is a second polarization direction, which differs from the first polarization direction.

4. A scanning endoscope device according to claim 1, further comprising a light-source controller that controls the illuminating light beam radiated from the first core portion and having the first optical characteristic and the illuminating light beam radiated from the second core portion and having the second optical characteristic so that the subject is irradiated simultaneously.

5. A scanning endoscope device according to claim 1, further comprising a light-source controller that controls the illuminating light beam radiated from the first core portion and having the first optical characteristic and the illuminating light beam radiated from the second core portion and having the second optical characteristic so that the subject is irradiated in time-division multiplexing fashion.

6. A scanning endoscope device according to claim 1, further comprising an optical component that is provided on the distal-end side of the first core portion and the second core portion to cause condensing of the illuminating light beams.

7. A scanning endoscope device according to claim 1, further comprising a control unit that synchronizes the driving unit and the image generating unit with each other so that images of the return light beams are formed in accordance with vibration of the first core portion and the second core portion caused by the driving unit.

8. A scanning endoscope device according to claim 1, wherein the driving unit causes the distal-end portion of the first core portion and the distal-end portion of the second core portion to vibrate together.
